(19) United States
(12) Patent Application Publication
(10) Pub. No.: US 2003/0175909 A1
(43) Pub. Date: Sep. 18, 2003

(54) Novel thermostable galactose isomerase and tagatose production thereby

(76) Inventors: Pil Kim, Seoul (KR); Sang Hyun Yoon, Seoul (KR); Myung Ji Seo, Seoul (KR); Jin Hwan Choi, Seoul (KR); Hoe Jin Roh, Seoul (KR)

Correspondence Address: BURNS DOANE SWECKER & MATHIS L L P, POST OFFICE BOX 1404, ALEXANDRIA, VA, 22313-1404, US

(21) Appl. No.: 10/204,220

(22) PCT Filed: Apr. 20, 2001
(86) PCT No.: PCT/KR01/00654

(30) Foreign Application Priority Data
Dec. 19, 2000 (KR) 2000/78833

(51) Int. Cl. C12P 21/02; C12N 1/21; C12N 5/06; C07H 21/04; C12P 19/24; C12N 9/90

(52) U.S. Cl. 435/94; 435/233; 536/23.2; 435/69.1; 435/320.1; 435/325

(57) ABSTRACT

Disclosed are novel thermostable galactose isomerases and production of tagatose using the same. A gene encoding a galactose isomerase with improved thermal stability and reaction equilibrium is screened from natural genetic materials. An expression vector into which the gene is inserted is introduced into bacteria which are then cultured to obtain a thermostable galactose isomerase. In the presence of this enzyme, tagatose is produced from galactose in a yield as high as 46-50% at a temperature as high as 55° C.

### TECHNICAL FIELD

The present invention relates, in general, to novel thermostable galactose isomerases and the production of tagatose using the same and, more particularly, to novel thermostable galactose isomerases with high enzymatic activity at high temperature and a method for producing tagatose from galactose in a high yield. Also, the present invention is concerned with genetic materials encoding the thermostable galactose isomerases, including genes, and expression vectors containing the genes.

### BACKGROUND ART

Tagatose, an isomer of galactose, is known to have almost the same sweetness as, and be the closest in sweetness quality to, fructose. Also, tagatose serves as a non-calorigenic sweetener because, when being ingested in the body, tagatose is neither metabolized, nor contributes to production of caloric values. Additionally, while sugar alcohols, the most prevalent sugar-substitutes in current use, have such a laxative effect that more than a certain intake of sugar alcohols causes diarrhea, tagatose enjoys the advantage of not having the laxative effect. Another advantage of tagatose is that, in contrast to sugar alcohols, tagatose can give appropriate flavors upon food processing because of its brown change upon heating like sugar. These properties have attracted great attention to tagatose as a sugar substitute with a great market potential (Zehener, 1988, EP 257626; Marzur, 1989, EP 0341062A2).

At present, D-tagatose is generally produced by chemical or biological methods. U.S. Pat. No. 4,273,922, yielded Jun. 16, 1981, refers to a chemical method for production of D-tagatose. According to the method, when a ketose is produced by adding boric acid to an aldose in the presence of a tertiary or quaternary amine, the boric acid and the ketose form a complex thereby to effectively move the reaction equilibrium toward ketose production. Another chemical method can be found in Korean Pat. No. 99-190671 which discloses that an aqueous galactose solution is isomerized by use of a metal hydroxide at pH 10 or higher at −15 to 40° C. in the presence of a soluble alkali metal salt or alkaline earth metal salt until insoluble precipitates consisting of metal hydroxide-tagatose complexes are formed. However, the conventional chemical methods are now evaluated to be insufficient for the mass production of tagatose. The chemical methods, although being acceptable in view of economics and production yield, are complicated as they are and must be conducted under specific conditions in addition to suffering from the disadvantage of being inefficient and producing industrial wastes.

For these reasons, biological methods, which are generally environmentally-friendly, are preferred unless they are economically unfavorable relative to chemical methods. Particularly when account is taken of environmental damage, biological processes which take advantage of microbes in producing tagatose from cheap carbohydrates obtainable from waste biological materials are very economically and environmentally favorable. Indeed, active research has been directed to such biological processes. Significant advance in the production of tagatose through biological processes was achieved by Izumori group, Japan. They developed a biological conversion method using galactitol dehydrogenase derived from an Arthrobacter strain, by which galactitol was converted to tagatose in a yield of 70-80% (Izumori and Tsuzuki, Production of D-tagatose from D-galactitol by Mycobacterium smegmalis, J. Ferment. Technol., 66, 225-227 (1988)). This conversion method, however, is problematic in that not only is the substrate galactitol expensive and difficult to secure in a large quantity, but also galactitol dehydrogenase requires expensive NAD (Nicotinamide-adenine dinucleotide) as a cofactor for its activity.

In the art, enzymatic processes for converting aldose or its derivatives into ketose or its derivatives are well known. For instance, production processes of fructose from glucose are extensively conducted on commercial scales. However, enzymatic processes for converting galactose to tagatose have not been actively used until recently.

The present inventors applied a method for the production of tagatose from galactose using E. coli-derived arabinose isomerase for applications (Korean Pat. Appl'n No. 99-16118; International Patent Appl'n No. PCT/KR99/00661) and reported that this method affords the conversion of galactose to tagatose in a yield of about 20% (Kim et al., High Production of D-Tagatose, a Potential Sugar Substitute, using Immobilized L-Arabinose Isomerase, Biotechnol. Prog. MS090-0400 (accepted); “Preparation of L-Arabinose Isomerase Originated from Escherichia coli as a Biocatalyst for D-Tagatose Production”, Biotechnol. Letts. 22 (3):197-199 (2000); “Bioconversion of D-Galactose to D-Tagatose by Expression of L-Arabinose Isomerase”, Biotechnol. Appl. Biochem., 31 (1): 1-4 (2000)). However, the enzyme suffers from the disadvantage of being poor in thermal stability and conversion yield.

Like glucose isomerase, arabinose isomerase exhibits different catalytic actions in vivo and in vitro, as shown in FIG. 2. Whereas it catalyzes the isomerization of arabinose to ribulose in vivo, arabinose isomerase acts in vitro to facilitate the conversion of galactose to tagatose. As in the reaction catalyzed by glucose isomerase, the equilibrium of the isomerization between galactose and tagatose, that is, between an aldose and a ketose, which is catalyzed by arabinose isomerase, varies with reaction temperature. The reaction proceeds toward the ketose as the reaction temperature increases. This has been already demonstrated in the production of fructose using glucose isomerase.

### DISCLOSURE OF THE INVENTION

Based on this background, the present inventors have searched for a novel galactose isomerase which can stably maintain enzymatic activity at high temperatures and shift the equilibrium of the whole reaction rates toward tagatose.

Leading to the present invention, the thorough and intensive research on a thermostable enzyme which can convert galactose to tagatose, conducted by the present inventors, resulted in the finding that a gene screened from some thermophilic microbes codes for a thermostable galactose isomerase in the presence of which galactose can be isomerized to tagatose in a high yield. In the present invention, a thermostable enzyme with arabinose isomerization activity was cloned and found to shift the equilibrium of the reaction rates between galactose and tagatose toward tagatose, and named “galactose isomerase”.

Therefore, it is an object of the present invention to provide a gene coding for a novel thermostable galactose isomerase, newly cloned from nature.

It is another object of the present invention to provide an amino acid sequence of the novel thermostable galactose isomerase, which can catalyze the conversion of galactose to tagatose in a high efficiency at high temperatures.

It is yet another object of the present invention to provide a genetically mutated gene encoding a novel thermostable galactose isomerase with greater catalytic activity compared to the above galactose isomerase.

It is a further object of the present invention to provide a recombinant expression vector which anchors a novel gene encoding the galactose isomerase therein.

It is still a further object of the present invention to provide a method for producing the galactose isomerase using a microbe transformed with the recombinant expression vector.

It is still another object of the present invention to provide a method for producing tagatose in a high production yield by use of the thermostable galactose isomerase.

Other objectives and advantages of the present invention will become apparent from the following detailed description.

### BRIEF DESCRIPTION OF THE DRAWINGS

FIG. 1 shows chemical structures of tagatose and galactose.

FIG. 2 shows different enzymatic functions of galactose isomerase and glucose isomerase between in vitro and in vivo.

FIG. 3 shows PCR primer sequences for priming in PCR for cloning galactose isomerase, consisting of six base sequences.

FIG. 4 is a UV photograph showing PCR products from naturally occurring thermophilic cells, separated by electrophoresis.

FIG. 5 shows a base sequence (Sequence No. 1) of the galactose isomerase gene cloned according to the present invention.

FIG. 6 shows an amino acid sequence (Sequence No. 2) of the galactose isomerase encoded by the gene.

FIG. 7 is a map of the recombinant expression vector pL151MO into which the gene encoding the galactose isomerase of the present invention is inserted.

FIG. 8 is a UV photograph showing restriction enzyme digests of pL151MO.

FIG. 9 shows an apparatus for the quantification of enzymatic activities of known arabinose isomerase and the galactose isomerase of the present invention.

FIG. 10 is a curve in which the relative activity of the galactose isomerase of the present invention is plotted versus reaction temperature.

FIG. 11 is a curve in which the relative activity of the galactose isomerase of the present invention is plotted versus pH.

FIG. 12 shows a genetically mutated base sequence (Sequence No. 6) of the galactose isomerase gene screened from the natural genetic resources.

### BEST MODES FOR CARRYING OUT THE INVENTION

The present invention is characterized in that a gene encoding galactose isomerase with improved thermal stability and reaction equilibrium is screened from natural genetic sources and tagatose is produced from galactose in the presence of the galactose isomerase which can be obtained in a large quantity from a transformant with an expression vector having the gene.

In the present invention, thermostable strains were isolated from hot spring areas. A DNA pool made from the isolated strains was used for PCR using consensus DNA fragments derived from the known base sequences for arabinose isomerase of three species Escherichia coli (Sequence No. 3), Bacillus subtilis (Sequence No. 4) and Salmonella typhimurium (Sequence No. 5). Three noticeable PCR products were subcloned into expression vectors which were introduced into host cells. In the transformants, the genes of interest were expressed as proteins which were then found to have enzymatic activity for galactose-tagatose conversion at high temperatures. From the clones with tagatose isomerization activity was prepared a gene encoding galactose isomerase, whose base sequence was found to share little homology with those of known arabinose isomerases. As for the amino acid sequence of the newly cloned gene, it has also little homology with known amino acid sequences. In detail, the new clone of the present invention shares homology of 9.5% in base sequence and 20.0% in amino acid sequence with E. coli, 61.6% in base sequence and 55.4% in amino acid sequence with Bacillus subtilis, and 58.5% in base sequence and 54.3% in amino acid sequence with Salmonella typhimurium. In addition, the isomerase of the present invention was found to stably perform the catalytic reaction even at 55° C. and exhibit a conversion yield of about 46-50%.

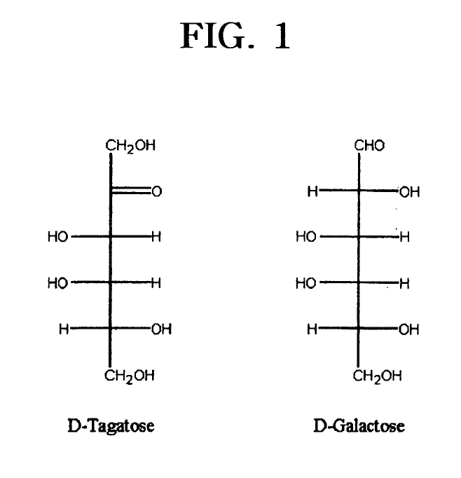

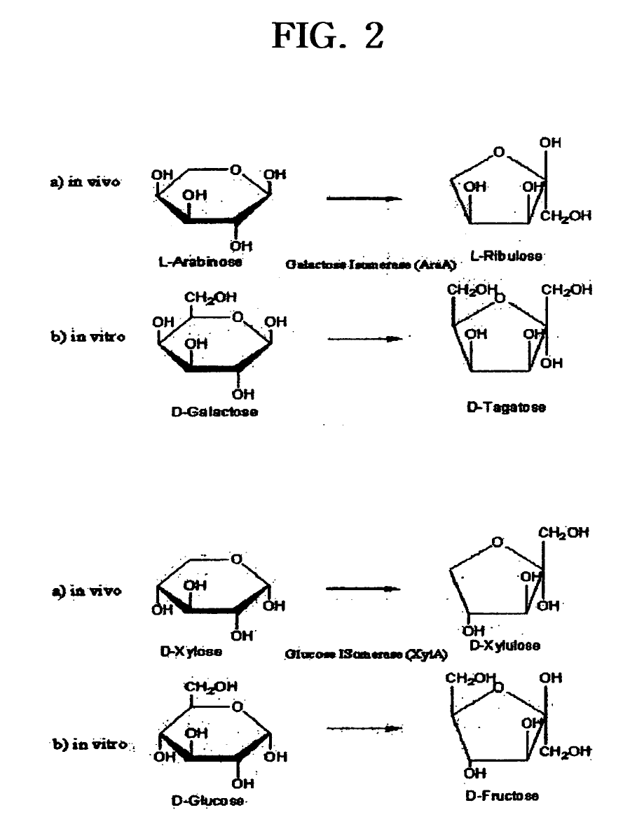

With reference to FIG. 1, there are chemical structures of two isomers D-tagatose and D-galactose. Like other galactose isomerases, the galactose isomerase of the present invention has different catalytic actions in vitro and in vivo conditions, as shown in FIG. 2. That is, the isomerase of the present invention converts galactose into tagatose in vitro while isomerizing arabinose into ribulose in vivo.

It can be inferred from the DNA sequence obtained that the amino acid expressed from the DNA sequence consists of 498 amino acids with a molecular weight of 56 kDa. An experiment revealed that the optimal reaction temperature and pH for the conversion of tagatose from galactose by the isomerase of the present invention were 60° C. and 7.5-8.5, respectively.

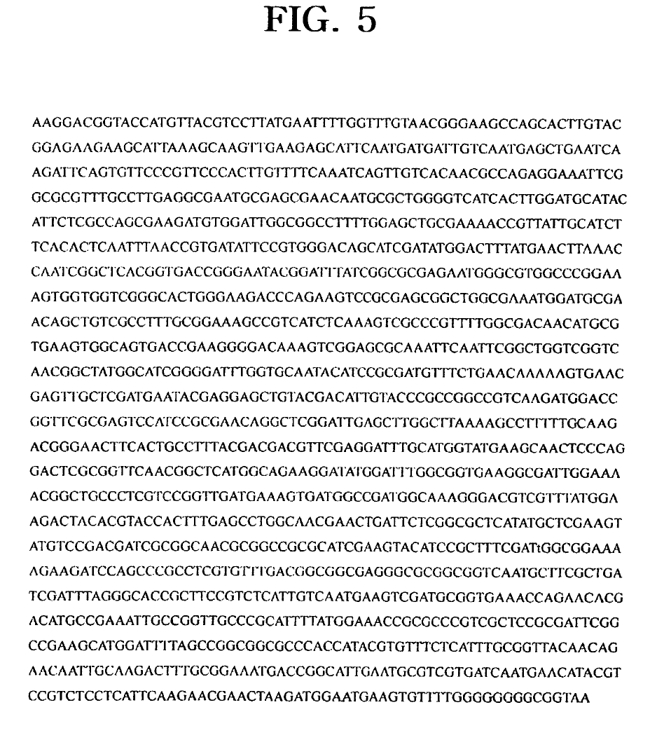

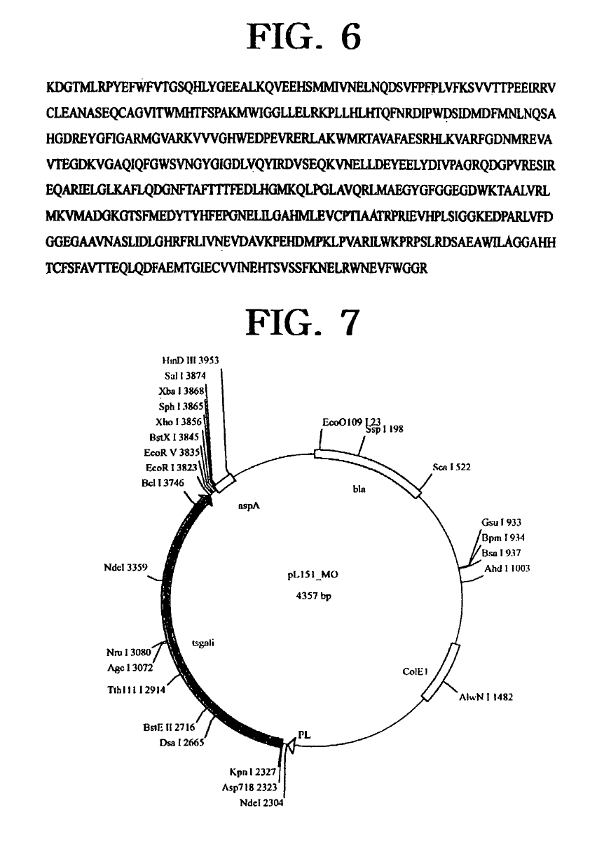

As will be explained in detail later, the isomerase with the conversion activity from galactose to tagatose was named “galactose isomerase”, whose base sequence (Sequence No. 1) and amino acid sequence (Sequence No. 2) are shown in FIGS. 5 and 6, respectively.

Herein, those who are skilled in the art should understand that the present invention deservedly comprises DNA or RNA sequences able to hybridize with the base sequence encoding the galactose isomerase of the present invention according to well-known techniques, such as those disclosed by Sambrook (Sambrook, et al., Molecular Cloning: A Laboratory Manual, 2ed. Vol. 1. pp. 101-104, Cold Spring Harbor Laboratory Press (1989)).

Therefore, it should be understood that the nucleic acid molecules interpreted by the present invention comprise those having base sequences inductively inferable from the base sequence or the amino acid sequence of the galactose isomerase obtained above as well as those having the base sequences hybridizable with the base sequence of the present invention or having the base sequences with codon degeneracy.

From the genes coding for the galactose isomerase of the present invention, various mutant enzymes which are modified in activity by in-vitro molecular evolution or directed evolution, both leading to artificial mutants, can be prepared. Techniques for constructing modified enzymes are known in the art and include, for example, chemical mutagenesis, error-prone PCR (mutagenic PCR), cassette mutagenesis, DNA suffling, etc. In a preferred embodiment of the present invention, gene mutagenesis was conducted through an error-prone PCR to afford a mutant enzyme with an activity 11 fold higher than that of the intact galactose isomerase obtained.

Herein, it should be noted that the present invention comprises a thermostable galactose isomerase as well as its amino acid sequence (Sequence No. 2) and also functionally equivalent molecules in which amino acid residues are substituted for residues within the sequence resulting in a silent change. For example, one or more amino acid residues within the intact sequence can be substituted by another amino acid(s) of a similar polarity which acts as a functional equivalent, resulting in a silent alteration. Substitutes for an amino acid within the sequence may be selected from other members of the class to which the amino acid belongs. For example, the class of nonpolar(hydrophobic) amino acids alanine, valine, leucine, isoleucine, phenylalanine, tryptophane, proline, and methionine. The polar neutral amino acids include glycine, serine, threonine, cystein, tyrosine, asparagines, and glutamine. The positively charged(basic) amino acids include arginine, lysine and histidine. The neagively charged(acidic) amino acids include aspartic acid and glutamic acid.

Also, included within the scope of the present invention are proteins or fragments or derivatives thereof which exibit the same and similar activity with amino acid homology of about 90-100% with the intact amino acid sequence (Sequence No. 2).

Additionally, isomerases which are equivalent in enzymatic activity or similar or identical in amino acid sequence to the galactose isomerase of the present invention may be derived from other microbes, such as E. coli, Bacillus sp., Salmonella sp., Enterobacter sp., Pseudomonas sp., Lactobacillus sp., Zymomonas sp., Gluconobacter sp., Rhizobium sp., Acetobacter sp., Rhodobacter sp., Agrobacterium sp., etc.

The present invention also encompasses the DNA with the nucleotide sequence that encodes the protein described herein as thermostable galactose isomerase, as well as expression vectors containing the DNA. One skilled in the art can prepare the recombinant expression vectors which comprise genes encoding the galactose isomerase or its mutants with transcription/translation regulatory sequences, using well-known cloning techniques. Any vector may be selected as the expression vector of the present invention if it is functional within selected host cells. For example, ordinary expression vectors, such as phages, plasmids, cosmids, etc., can be utilized. Construction methods of expression vectors are well known and disclosed in detail in, for example, Sambrook et al., Molecular Cloning, Cold Spring Harbor Laboratory (1989).

Using the recombinant expression vectors thus prepared, host cells may be transformed. Available as host cells for the recombinant DNA are various cells, including bacteria, Actinomycetes, yeast, fungi, animal cells, insect cells and plant cells.

After being transformed with a recombinant expression vector which anchors a gene encoding the thermostable galactose isomerase of the present invention or a functional equivalent, a host cell is cultured in a suitable medium under appropriate conditions to produce galactose isomerase.

Under appropriate conditions, the galactose isomerase prepared according to the present invention can be used to convert galactose to tagatose. In this regard, the enzyme may be in a free state or may be immobilized to a suitable carrier.

In an embodiment of the present invention, the gene encoding the thermostable galactose isomerase, cloned from the thermophilic strains by PCR technique, was introduced to E. coli. After being cultured, the cells harboring the gene were lysed to obtain the cytosol as an enzyme source. Then, the enzyme source was added to a buffer (pH 7.0) containing galactose 5 g/l and reacted at 55° C. For comparison, E. coli JM105 and E. coli transformed with the recombinant pTC101 containing araA of E. coli (Korean Pat. No. Appl'n No. 99-16118; International Pat. Appl'n No. PCT/KR99/00661) were lysed to obtain cytosols which were then used to convert galactose, as in the cytosol containing the galactose isomerase of the present invention. As a result, the arabinose isomerase derived from E. coli showed almost no catalytic activity at such high temperatures while the novel thermostable galactose isomerase of the present invention actively catalyzed the conversion of tagatose from galactose at the high temperature. Additionally, the production yield was found to be 48%, which was much increased compared to the production yield of 30% obtainable when tagatose was produced form galactose in the presence of the E. coli-derived arabinose isomerase at room temperature.

Tagatose, which can be produced from galactose in a high yield in the presence of the galactose isomerase of the present invention, has numerous applications in various fields, for example, including sweeteners for low caloric foods, fillers, intermediates for synthesizing optically active compounds, and an additive of detergents, cosmetics, and pharmaceuticals.

Thanks to improved thermal stability, the galactose isomerase of the present invention can catalyze the conversion of tagatose from galactose at high temperatures, thereby directing the reaction equilibrium therebetween toward tagatose. Unlike conventional chemical methods, the biological method for producing tagatose by use of the thermostable isomerase according to the present invention is environmentally friendly. Further to these, the biological method of the present invention can reduce the production cost greatly compared to conventional methods using galactitol dehydrogenase because galactose is cheaper than galactitol.

A better understanding of the present invention may be obtained in light of the following examples which are set forth to illustrate, but are not to be construed to limit the present invention.

### EXAMPLE 1

### Preparation of Genomic Libraries from Thermophilic Bacteria

To obtain genomic libraries of thermophilic bacteria, soil samples were taken from hot spring areas at Samchuk, Korea. Suspensions of the soil samples in distilled water were spread on LB media without dilution, followed by incubation at 55° C. After 24 hours of incubation, the thermophilic cells appearing as colonies on the media were inoculated in liquid LB media and cultured again for 12 hours at the same temperature. From a pool of the thermophilic cells thus obtained, a genomic library was prepared according to the instruction disclosed in Sambrook et al., Molecular Cloning: A Laboratory Manual 2^{nd} Ed., Cold Spring Harbor Laboratory Press (1989).

### EXAMPLE 2

### Screening and Cloning of Galactose Isomerase from Genomic Library of Thermophilic Bacteria

First Step: PCR of Galactose Isomerase Gene and Construction of Recombinant Expression Vector

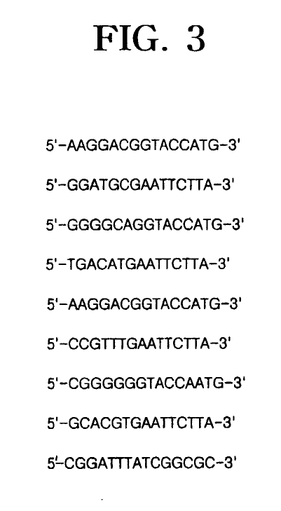

The genomic library prepared from the thermophilic bacteria in Example 1 was screened by PCR to find a gene encoding galactose isomerase. Primers used for the PCR were synthesized under the design concept that each of them must contain a consensus part of araA base sequences of E. coli, B. subtilis, and S. typhimurium, and at least one restriction enzyme site for subcloning, and consist of 15 bases or less. As shown in FIG. 3, the synthesized primers had the following sequences:

5′-AAGGACGGTACCATG-3′; 5′-GGATGCGAATTCTTA-3′;
5′-GGGGCAGGTACCATG-3′; 5′-TGACATGAATTCTTA-3′;
5′-AAGGACGGTACCATG-3′; 5′-CCGTTTGAATTCTTA-3′;
5′-CGGGGGGTACCAATG-3′; 5′-GCACGTGAATTCTTA-3′;
5′-CGGATTTATCGGCGC-3′; 5′-CTTATGCCATGAGCC-3′;
5′-TCGCCGCCGTCAAAC-3′; and 5′-GACAAGTTTGATATT-3′.

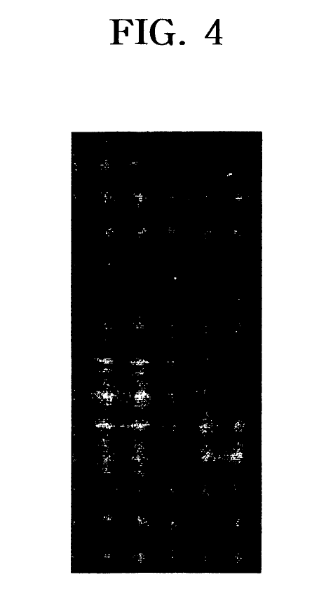

As for the PCR, its annealing temperature was set at as low as 45° C. not to impair the association possibility in non-specific regions. PCR was carried out in a thermal cycler, with cycles of denaturation at 96° C. for 30 sec, annealing at 45° C. for 30 sec and polymerization at 72° C. for 3 min, so as to produce DNA fragments which were found to be 1.5, 2.5 and 4 kb in size, respectively, as measured through gel electrophoresis (FIG. 4).

Each PCR product was ligated to pLEX, a 2.9 kb expression vector (Invitrogen, USA), with which E. coli JM105 was transformed.

Second Step: Transformants Screening

After the transformation of the First Step, E. coli JM105 was spread over agar plates containing ampicillin as a selection marker. Colonies grown with ampicillin resistance were counted to about 150. After being cultured in liquid media, the cells were harvested, and lysed with the aid of an ultrasonic processor to obtain cytosols. Each of the cytosols obtained was added to a galactose-containing buffer and reacted for 24 hours at 55° C. Detection of tagatose enabled the selection of the clones that harbored a galactose isomerase gene.

### EXAMPLE 3

### Determination of Base Sequence of Cloned Galactose Isomerase Gene and its Encoded Amino Acid Sequence

After being prepared from the selected clones, the expression vector was digested with restriction enzymes (EcoRI-KpnI). The DNA fragment obtained was sequenced, followed by the determination of its amino acid sequence deduced from the base sequence thus determined. The base and amino acid sequences of the galactose isomerase gene( [symbol] ) are given in FIGS. 5 and 6, respectively.

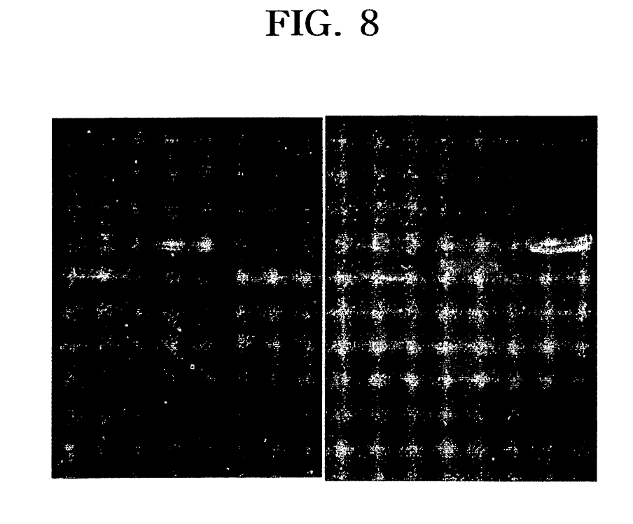

The vector containing the gene encoding the thermostable galactose isomerase was named “pL151MO” and its genetic map is given as shown in FIG. 7. The recombinant expression vector pL151MO was cut with the restriction enzymes, after which the digests were determined for size by gel electrophoresis as shown in FIG. 8.

### EXAMPLE 4

### Production of Tagatose in Galactose Media

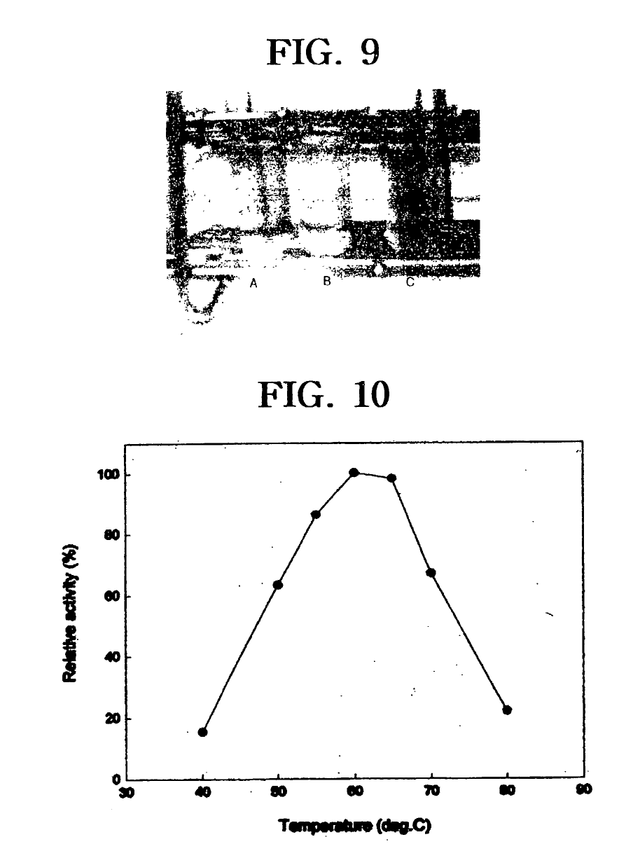

The E. coli JM105/pL151MO harboring the gene of the novel thermostable galactose isomerase obtained in Example 2, was tested, along with other control groups, for the production of tagatose from galactose at high temperatures. As the control groups, intact E. coli JM105 and E. coli transformed with the recombinant expression vector pTC101 containing araA of E. coli (Korean Pat. Appl'n No. 99-16118; International Pat. Appl'n No. PCR/KR99/00661) were used. After culturing the E. coli species, each of the biomasses thus obtained was lysed to obtain an enzyme source. To a pH 7.0 buffer containing galactose 5 g/l was added the cytosolic lysate, followed by reacting at 55° C. After 12 hours of the reaction, the tagatose produced was quantified by a coloring method using cystein-carbazole. These results are shown in FIG. 9. Quantification of the produced tagatose was also conducted after 72 hours and the resulting amounts are given in Table 1, below.

TABLE 1
Amounts of Tagatose Produced by the Thermostable Galactose
Isomerase and Conventional Enzymes

| Strain | Tagatose Produced (g/l) |
|---|---|
| JM105 | 0 |
| JM105/pTC101 | 0.1 |
| JM105/pL151MO | 2.4 |

As apparent from Table 1, the novel thermostable galactose isomerase of the present invention showed enzymatic activity at high temperatures while the arabinose isomerase derived from E. coli produced almost no tagatose at high temperatures. Also, the yield of the reaction between galactose-tagatose was as high as about 48% at such high temperatures in the presence of the enzyme of the present invention. This was significantly increased compared to the production yield obtainable from the E. coli-derived arabinose isomerase, which was measured to be only 30%. The increase in equilibrium constant attributed to the thermal stability of the galactose isomerase agrees with the case of glucose isomerase (Bhosale et al., Molecular and industrial aspects of glucose isomerase, Microbiol. Rev., 60:280-300), demonstrating that the equilibrium constant between aldoses and ketoses is dependent on temperature, as in general reactions.

### EXAMPLE 5

### Determination of Optimal Reaction Temperature and Optimal pH

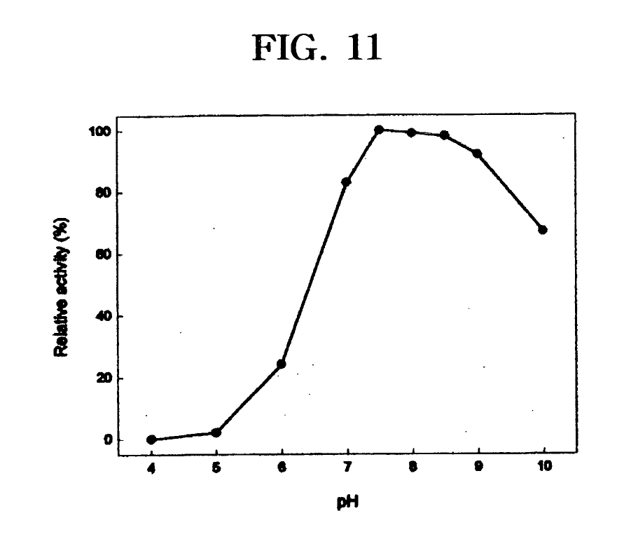

From the DNA sequence determined, the galactose isomerase of the present invention could be inferred to consist of 498 amino acid residues with a molecular weight of 56 kDa. An examination was made of the determination of optimal reaction temperature and pH, in which relative activity of the enzyme was measured while varying temperature and pH. Changes in activity with regard to temperature and pH are shown in FIGS. 10 and 11, respectively, indicating that the optimal reaction temperature and pH of the enzyme is 60° C. and 7.5-8.5, respectively.

### EXAMPLE 6

### Molecular Evolution of Galactose Isomerase Gene

Modification was performed for the gene encoding the thermostable galactose isomerase by use of an error-prone PCR method. While the intact gene of the galactose isomerase serves as a template, an error-prone PCR was conducted. After digestion with restriction enzymes, the PCR product was subcloned into the vector pKK223-3 (AP Biotech, Genbank: M77749). Selection of subclones was carried out on LB-agar plates containing ampicillin. Colonies which were grown on the plates were transferred to 96-well plates and cultured in galactose (1%) media at 60° C. for an additional 6 hours. Each well was visualized by treatment with cystein-carbazole, followed by measuring the absorbance at 560 nm with aid of an ELISA reader. Selected were the colonies which were increased in activity compared to colonies containing pL151MO. Of 1,000 colonies selected, 6 colonies were found to show catalytic activities which were highly increased compared to the intact galactose isomerase. The results are given in Table 2, below.

TABLE 2
Activities of Intact and Mutant Galactose Isomerases

| | pL151MO | A3 | B7 | E10 | C4 | G5 | F9 |
|---|---|---|---|---|---|---|---|
| Abs. of cell | 0.79 | 0.92 | 0.37 | 0.56 | 0.56 | 0.69 | 1.17 |
| Abs. of Tagatose | 0.042 | 0.554 | 0.158 | 0.038 | 0.124 | 0.144 | 0.341 |
| Abs. Ratio | 5.3 | 60.4 | 43.1 | 6.8 | 22.0 | 20.7 | 29.2 |
| (tagatose/cell) | | | | | | | |
| Folds | 1 | 11.4 | 8.1 | 1.3 | 4.2 | 3.9 | 5.5 |

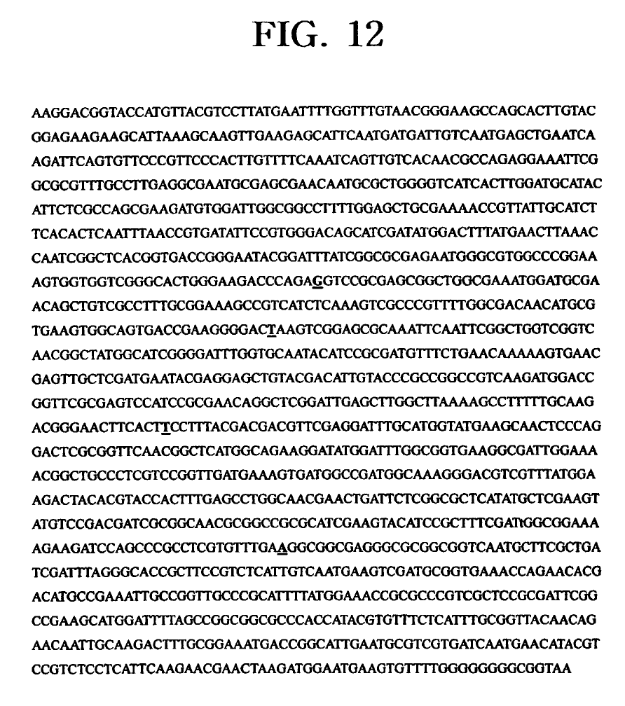

A mutant galactose isomerase showed activity as high as 11 times greater than that of intact galactose isomerase. From the colony with the highest activity was prepared the plasmid which was then used to determine the base sequence of the mutant gene of interest. The result is given in FIG. 12. In the base sequence, substituted bases are underlined.

### INDUSTRIAL APPLICABILITY

As described hereinbefore, the thermostable galactose isomerase of the present invention and its mutants have such high enzymatic activities as to produce tagatose from galactose at high temperatures in high yields.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A gene having the base sequence of Sequence No. 1, encoding a thermostable galactose isomerase, or having a base sequence with codon degeneracy, encoding a functional equivalent to the thermostable galactose isomerase.

2. A gene having a base sequence encoding a thermostable galactose isomerase, which shares homology of 95% or greater with the gene of claim 1, or having a base sequence with codon degeneracy, encoding a functional equivalent to the thermostable galactose isomerase.

3. The gene as set forth in claim 2, wherein the base sequence is Sequence No. 6.

4. A thermostable galactose isomerase protein having the amino acid sequence of Sequence No. 2, or a derivative thereof having an amino acid sequence in which some amino acid residues are substituted with functionally identical or similar amino acid residues.

5. A thermostable galactose isomerase protein having an amino acid sequence sharing a homology of 95% or higher with the amino acid sequence of claim 4, or a derivative thereof having an amino acid sequence in which some amino acid residues are substituted with functionally identical or similar amino acid residues.

6. A recombinant expression vector, containing a gene of any of claims 1 to 3.

7. A cell strain, transformed with the recombinant expression vector of claim 6.

8. A method for preparing a thermostable galactose isomerase, in which the transformed cell is cultured.

9. A method for producing tagatose from galactose, in which the thermostable galactose isomerase of claim 4 or 5 or the thermostable galactose isomerase produced according to the method of claim 8 is used.


SEQUENCE LISTING (6 sequences)

SEQ ID NO: 1 - DNA, 1497 bp, Unknown
  Galactose isomerase
aaggacggtaccatgttacgtccttatgaattttggtttgtaacgggaagccagcacttgtacggagaagaagcattaaagcaagttgaagagcattcaatgatgattgtcaatgagctgaatcaagattcagtgttcccgttcccacttgttttcaaatcagttgtcacaacgccagaggaaattcggcgcgtttgccttgaggcgaatgcgagcgaacaatgcgctggggtcatcacttggatgcatacattctcgccagcgaagatgtggattggcggccttttggagctgcgaaaaccgttattgcatcttcacactcaatttaaccgtgatattccgtgggacagcatcgatatggactttatgaacttaaaccaatcggctcacggtgaccgggaatacggatttatcggcgcgagaatgggcgtggcccggaaagtggtggtcgggcactgggaagacccagaagtccgcgagcggctggcgaaatggatgcgaacagctgtcgcctttgcggaaagccgtcatctcaaagtcgcccgttttggcgacaacatgcgtgaagtggcagtgaccgaaggggacaaagtcggagcgcaaattcaattcggctggtcggtcaacggctatggcatcggggatttggtgcaatacatccgcgatgtttctgaacaaaaagtgaacgagttgctcgatgaatacgaggagctgtacgacattgtacccgccggccgtcaagatggaccggttcgcgagtccatccgcgaacaggctcggattgagcttggcttaaaagcctttttgcaagacgggaacttcactgcctttacgacgacgttcgaggatttgcatggtatgaagcaactcccaggactcgcggttcaacggctcatggcagaaggatatggatttggcggtgaaggcgattggaaaacggctgccctcgtccggttgatgaaagtgatggccgatggcaaagggacgtcgtttatggaagactacacgtaccactttgagcctggcaacgaactgattctcggcgctcatatgctcgaagtatgtccgacgatcgcggcaacgcggccgcgcatcgaagtacatccgctttcgattggcggaaaagaagatccagcccgcctcgtgtttgacggcggcgagggcgcggcggtcaatgcttcgctgatcgatttagggcaccgcttccgtctcattgtcaatgaagtcgatgcggtgaaaccagaacacgacatgccgaaattgccggttgcccgcattttatggaaaccgcgcccgtcgctccgcgattcggccgaagcatggattttagccggcggcgcccaccatacgtgtttctcatttgcggttacaacagaacaattgcaagactttgcggaaatgaccggcattgaatgcgtcgtgatcaatgaacatacgtccgtctcctcattcaagaacgaactaagatggaatgaagtgttttggggggggcggtaa

SEQ ID NO: 2 - protein, 498 aa, Unknown
  Galactose isomerase
KDGTMLRPYEFWFVTGSQHLYGEEALKQVEEHSMMIVNELNQDSVFPFPLVFKSVVTTPEEIRRVCLEANASEQCAGVITWMHTFSPAKMWIGGLLELRKPLLHLHTQFNRDIPWDSIDMDFMNLNQSAHGDREYGFIGARMGVARKVVVGHWEDPEVRERLAKWMRTAVAFAESRHLKVARFGDNMREVAVTEGDKVGAQIQFGWSVNGYGIGDLVQYIRDVSEQKVNELLDEYEELYDIVPAGRQDGPVRESIREQARIELGLKAFLQDGNFTAFTTTFEDLHGMKQLPGLAVQRLMAEGYGFGGEGDWKTAALVRLMKVMADGKGTSFMEDYTYHFEPGNELILGAHMLEVCPTIAATRPRIEVHPLSIGGKEDPARLVFDGGEGAAVNASLIDLGHRFRLIVNEVDAVKPEHDMPKLPVARILWKPRPSLRDSAEAWILAGGAHHTCFSFAVTTEQLQDFAEMTGIECVVINEHTSVSSFKNELRWNEVFWGGR

SEQ ID NO: 3 - DNA, 1532 bp, Escherichia coli
  gene (1)...(1532): Arabinose isomerase
atgcggctacttagcgacgaaacccgtaatacacttcgttccagcgcagcgcgtctttaaacgctggcaggcgtgtgtcgttatcaatcaccgtgatttcaatgtcgtgcatctcggcgaattggcgcatatcgttgaggttcagtgcatggctgaagacggtatggtgcgcgccaccagcgaggatccacgcttcggaagcagttggcagatccggttgcgctttccacagcgcattcgccaccggcagtttcggcagggagtgcggtgttttcaccgtgtcgatgcagttaaccagtagacggtaacgatcgccgagatcaatcaagctggcgacaatcgctgggccggtttgggtattgaagatcaggcgggcaggatcgtccttaccaccaataccgagatgctgaacgtcgaggatcggtttctcttctgcggcgatcgacgggcagacttccagcatatgggagccgagcaccaggtcattacctttctcgaagtgataggtgtagtcctccataaaggaggtgccgccctgcagaccggttgacatcaccttcatgatgcgaagcagggcggcagttttccagtcgccttcgcccgcaaagccgtaaccctgctgcatcagacgctgtacggccagaccaggaagctgtttcagaccgtgcaaatcttcaaaggtggtggtgaacgcgtggaagccaccttgttccaggaaacgcttcatccccagctcaatacgcgccgcttccagcacgttctgtcgttttttgccgtggatttgtgtggcaggcgtcatggtgtagcagctttcgtactcatcgaccagcgcgttaacatcgccgtcgctgatggagttcaccacctgcaccagatcgccaaccgcccaggtattgacggagaaaccgaacttgatctgtgcggcaactttatcgccatcggtgaccgccacttcacgcatgttatcgccaaatcggcagactttcagatgacgggtatcctgtttagagaccgcctgacgcatccaggagccgatacgctcatgggcttgtttatcctgccagtgaccggtaaccacggcatgttgctgacgcatacgcgcgccaatgaagccgaactcgcgaccgccatgtgcagtctggttcaggttcataaagtccatatcgatactgtcccacggcagcgccgcgttgaactgggtgtggaattgcagcaacggtttgttgagcatggtcaggccgttgatccacattttggccggggagaaggtgtgcagccacaccaccagaccagcgcaacgatcgtcgtaattcgcgtcgcggcaaatagcggtgatttcatccggcgtggtgcccagcggtttcaacaccagtttgcagggcagtttcgcttccgtattcagcgcattaacgacgtgctcggcatgttgggtgacctgacgcagggtttccgggccatacagatgctggctgccaatgacaaaccacacttcataattatcaaaaatcgtcattatcgtgtccttatagagt

SEQ ID NO: 4 - DNA, 1497 bp, Bacillus subtilis
  gene (1)...(1497): Arabinose isomerase
atgcttcagacaaaggattatgaattctggtttgtgacaggaagccagcacctatacggggaagagacgctggaactcgtagatcagcatgctaaaagcatttgtgaggggctcagcgggatttcttccagatataaaatcactcataagcccgtcgtcacttcaccggaaaccattagagagctgttaagagaagcggagtacagtgagacatgtgctggcatcattacatggatgcacacattttccccctcccaaaaattgtggaaaagaaggcctttccctccttatcaaaaaccgcttatgcatttgcatacccaatataatcgcgatatcccgtggggtacgattgacatggattttatgaacagcaaccaatccgcgcatggcgatcgagagtacggttacatcaactcgagaatggggcttagccgaaaagtcattgccggctattgggatgatgaagaagtgaaaaaagaaatgtcccagtggatggatacggcggctgcattaaatgaaagcagacatattaaggttgccagatttggagataacatgcgtcatgtcgcggtaacggacggagacaaggtgggagcgcatattcaatttggctggcaggttgacggatatggcatcggggatctcgttgaagtgatggatcgcattacggacgacgaggttgacacgctttatgccgagtatgacagactatatgtgatcagtgaggaaacaaaacgtgacgaagcaaaggtagcgtccattaaagaacaggcgaaaattgaacttggattaaccgcttttcttgagcaaggcggatacacagcgtttacgacatcgtttgaagtgctgcacggaatgaaacagctgccgggacttgccgttcagcgcctgatggagaaaggctatgggtttgccggtgaaggagattggaagacagcggcccttgtacggatgatgaaaatcatggctaaaggaaaaagaacttccttcatggaagattacacgtaccattttgaaccgggaaatgaaatgattctgggctctcacatgcttgaagtgtgtccgactgtcgctttggatcagccgaaaatcgaggttcattcgctttcgattggcggcaaagaggaccctgcgcgtttggtatttaacggcatcagcggttctgccattcaagctagcattgttgatattggcgggcgtttccgccttgtgctgaatgaagtcaacggccaggaaattgaaaaagacatgccgaatttaccggttgcccgtgttctctggaagccggagccgtcattgaaaacagcagcggaggcatggattttagccggcggtgcacaccatacctgcctgtcttatgaactgacagcggagcaaatgcttgattgggcggaaatggcgggaatcgaaagtgttctcatttcccgtgatacgacaattcataaactgaaacacgagttaaaatggaacgaggcgctttaccggcttcaaaagtag

SEQ ID NO: 5 - DNA, 1524 bp, Salmonella typhimurium
  gene (1)...(1524): Arabinose isomerase
atgacgatttttgataattatgaagtatggtttgtgattggcagccagcatttgtatggcgcagaaaccctgcgtcaggtcacccaacatgccgagcatgtggtcaacgcgctgaataccgaagccaaactgccatgtaaactggtattaaaaccgctgggcacctcgccggatgagattaccgccatttgtcgtgacgccaattatgacgatcgctgcgcagggctggtggtctggctgcacaccttctccccggccaaaatgtggatcaacgggctgagtatccttaacaaaccactactgcaattccatacccaatttaacgccgccctgccgtgggacagcattgatatggactttatgaacctgaaccagactgcgcacggcggtcgtgagttcggttttatcggcgcgcggatgcgccagcagcacgcggtcgtcaccggtcactggcaggataaagaggcccatacgcgtatcggtgcctggatgcgccaggcggtctctaaacaggatacccgccagctaaaagtctgccgcttcggcgacaatatgcgtgaagtcgcagtgactgacggtgataaagtggccgcgcaaatcaaatttggcttttcggtcaatacctgggcggtcggcgatctggtgcaggtggtgaattctatcggcgacggcgatatcaacgctctgattgacgagtatgaaagcagctataccctgacgcccgccacccaaatccacggcgataaacgccagaacgtgcgggaggcggcgggtattgaactcggtatgaagcgtttcctggaacagggcggcttccacgcattcactactacctttgaagatttacacggtctgaaacagcttccgggtctggccgtacagcgtctgatgcagcaaggctacggctttgcgggcgaaggcgactggaaaaccgccgctctgcttcgcattatgaaagtgatgtcaaccggtctgcagggcggcacctcatttatggaggattacacctaccacttcgagaaaggcaacgatctggtgctcggctcgcacatgctggaagtgtgtccgtccatcgcggtggaagagaaaccgatcctcgacgtccagcacctcggcattggcggcaaggaagatccggcgcgtttgattttcaatacccaaaccggcccggcgatcgtcgccagcctgatcgacctcggcgatcgttatcgcctgctggtcaactgcattgacaccgtaaaaacgccgcactccctgccgaaactgccggtgcgtaacgcgctgtggaaggcgcagccggatctgccgaccgcctccgaagcgtggattctggctggcggcgcgcaccataccgtcttcagccacgcgctggatctgaacgatatgcgccagtttgcagaaatacacgatatcgaaatcgcggtgattgataacgatacccatctgccggcctttaaggacgcgctgcgctggaacgaggtgtattacgggttcaaacgttaattggtgaaacggattgcctgg

SEQ ID NO: 6 - DNA, 1497 bp, Unknown
  Mutated galactose isomerase gene from sequence 1
aaggacggtaccatgttacgtccttatgaattttggtttgtaacgggaagccagcacttgtacggagaagaagcattaaagcaagttgaagagcattcaatgatgattgtcaatgagctgaatcaagattcagtgttcccgttcccacttgttttcaaatcagttgtcacaacgccagaggaaattcggcgcgtttgccttgaggcgaatgcgagcgaacaatgcgctggggtcatcacttggatgcatacattctcgccagcgaagatgtggattggcggccttttggagctgcgaaaaccgttattgcatcttcacactcaatttaaccgtgatattccgtgggacagcatcgatatggactttatgaacttaaaccaatcggctcacggtgaccgggaatacggatttatcggcgcgagaatgggcgtggcccggaaagtggtggtcgggcactgggaagacccagaggtccgcgagcggctggcgaaatggatgcgaacagctgtcgcctttgcggaaagccgtcatctcaaagtcgcccgttttggcgacaacatgcgtgaagtggcagtgaccgaaggggactaagtcggagcgcaaattcaattcggctggtcggtcaacggctatggcatcggggatttggtgcaatacatccgcgatgtttctgaacaaaaagtgaacgagttgctcgatgaatacgaggagctgtacgacattgtacccgccggccgtcaagatggaccggttcgcgagtccatccgcgaacaggctcggattgagcttggcttaaaagcctttttgcaagacgggaacttcacttcctttacgacgacgttcgaggatttgcatggtatgaagcaactcccaggactcgcggttcaacggctcatggcagaaggatatggatttggcggtgaaggcgattggaaaacggctgccctcgtccggttgatgaaagtgatggccgatggcaaagggacgtcgtttatggaagactacacgtaccactttgagcctggcaacgaactgattctcggcgctcatatgctcgaagtatgtccgacgatcgcggcaacgcggccgcgcatcgaagtacatccgctttcgattggcggaaaagaagatccagcccgcctcgtgtttgaaggcggcgagggcgcggcggtcaatgcttcgctgatcgatttagggcaccgcttccgtctcattgtcaatgaagtcgatgcggtgaaaccagaacacgacatgccgaaattgccggttgcccgcattttatggaaaccgcgcccgtcgctccgcgattcggccgaagcatggattttagccggcggcgcccaccatacgtgtttctcatttgcggttacaacagaacaattgcaagactttgcggaaatgaccggcattgaatgcgtcgtgatcaatgaacatacgtccgtctcctcattcaagaacgaactaagatggaatgaagtgttttggggggggcggtaa